SEQUENCE LISTING (88 sequences)

SEQ ID NO: 1 - RNA, 22 nt, Homo sapiens
  source 1..22: mol_type = unassigned RNA
tcaccgggtgtaaatcagcttg

SEQ ID NO: 2 - RNA, 79 nt, Homo sapiens
  source 1..79: mol_type = unassigned RNA
cccgggctgaggtaggaggttgtatagttgaggaggacacccaaggagatcactatacggcctcctagctttccccagg

SEQ ID NO: 3 - RNA, 23 nt, Homo sapiens
  source 1..23: mol_type = unassigned RNA
cctgcagcgacttgatggcttcc

SEQ ID NO: 4 - RNA, 111 nt, Homo sapiens
  source 1..111: mol_type = unassigned RNA
ccacggtcctagttaaaaaggcacattcctagaccctgcctcagaactactgaacagagtcactgggtgtggagtccaggaatctgcatttttacccctatcgcccccgcc

SEQ ID NO: 5 - RNA, 22 nt, Homo sapiens
  source 1..22: mol_type = unassigned RNA
tcaaaactgaggggcattttct

SEQ ID NO: 6 - RNA, 22 nt, Homo sapiens
  source 1..22: mol_type = unassigned RNA
gctatttcacgacaccagggtt

SEQ ID NO: 7 - RNA, 22 nt, Homo sapiens
  source 1..22: mol_type = unassigned RNA
ctcctacatattagcattaaca

SEQ ID NO: 8 - RNA, 22 nt, Homo sapiens
  source 1..22: mol_type = unassigned RNA
ccaatattactgtgctgcttta

SEQ ID NO: 9 - RNA, 23 nt, Homo sapiens
  source 1..23: mol_type = unassigned RNA
aacattcattgctgtcggtgggt

SEQ ID NO: 10 - RNA, 21 nt, Homo sapiens
  source 1..21: mol_type = unassigned RNA
ctatatatcaaacatattcct

SEQ ID NO: 11 - RNA, 21 nt, Homo sapiens
  source 1..21: mol_type = unassigned RNA
atgacctatgaattgacagac

SEQ ID NO: 12 - RNA, 22 nt, Homo sapiens
  source 1..22: mol_type = unassigned RNA
acctggcatacaatgtagattt

SEQ ID NO: 13 - RNA, 24 nt, Homo sapiens
  source 1..24: mol_type = unassigned RNA
agcgcgggctgagcgctgccagtc

SEQ ID NO: 14 - RNA, 21 nt, Homo sapiens
  source 1..21: mol_type = unassigned RNA
atccccagatacaatggacaa

SEQ ID NO: 15 - RNA, 22 nt, Homo sapiens
  source 1..22: mol_type = unassigned RNA
tggtttaccgtcccacatacat

SEQ ID NO: 16 - RNA, 23 nt, Homo sapiens
  source 1..23: mol_type = unassigned RNA
ctgactgaataggtagggtcatt

SEQ ID NO: 17 - RNA, 86 nt, Homo sapiens
  source 1..86: mol_type = unassigned RNA
gagggaaagcaggccaacctcgaggatctccccagccttggcgttcaggtgctgaggagatcgtcgaggttggcctgcttcccctc

SEQ ID NO: 18 - RNA, 85 nt, Homo sapiens
  source 1..85: mol_type = unassigned RNA
ggacctgccctgggctttctagtctcagctctcctccagctcagctggtcaggagagctgagactagaaagcccagggcaggttc

SEQ ID NO: 19 - RNA, 81 nt, Homo sapiens
  source 1..81: mol_type = unassigned RNA
acctgccctgggctttctagtctcagctctcctgaccagctgagctggaggagagctgagactagaaagcccagggcaggt

SEQ ID NO: 20 - RNA, 85 nt, Homo sapiens
  source 1..85: mol_type = unassigned RNA
ggcgcctcctgctctgctgtgccgccagggcctcccctagcgcgccttctggagaggctttgtgcggatacggggctggaggcct

SEQ ID NO: 21 - RNA, 79 nt, Homo sapiens
  source 1..79: mol_type = unassigned RNA
aattaatccctctctttctagttcttcctagagtgaggaaaagctgggttgagagggcaaacaaattaactaattaatt

SEQ ID NO: 22 - RNA, 23 nt, Homo sapiens
  source 1..23: mol_type = unassigned RNA
tcaagagcaataacgaaaaatgt

SEQ ID NO: 23 - RNA, 98 nt, Homo sapiens
  source 1..98: mol_type = unassigned RNA
acccaaaccctaggtctgctgactcctagtccagggctcgtgatggctggtgggccctgaacgaggggtctggaggcctgggtttgaatatcgacagc

SEQ ID NO: 24 - RNA, 23 nt, Homo sapiens
  source 1..23: mol_type = unassigned RNA
taggcagtgtcattagctgattg

SEQ ID NO: 25 - RNA, 24 nt, Homo sapiens
  source 1..24: mol_type = unassigned RNA
acaaaaaaaaaagcccaacccttc

SEQ ID NO: 26 - RNA, 92 nt, Homo sapiens
  source 1..92: mol_type = unassigned RNA
tgtcactccgccagcatcatgaagtgcactcatgatatgtttgccccatcagcgtgtcacgagggcatttcatgatgcaggcggggttggca

SEQ ID NO: 27 - RNA, 22 nt, Homo sapiens
  source 1..22: mol_type = unassigned RNA
ctgggaggtgtgatatcgtggt

SEQ ID NO: 28 - RNA, 19 nt, Homo sapiens
  source 1..19: mol_type = unassigned RNA
actggacttggaggcagaa

SEQ ID NO: 29 - RNA, 111 nt, Homo sapiens
  source 1..111: mol_type = unassigned RNA
cttttgctgtcagtttttctgttgcttgtcttggttttatgccttttatatcaaggcacataaaaggcataaaaccaagacaagcaacaaaaaaaggattgatcacagaag

SEQ ID NO: 30 - RNA, 22 nt, Homo sapiens
  source 1..22: mol_type = unassigned RNA
tcaggtgtggaaactgaggcag

SEQ ID NO: 31 - RNA, 17 nt, Homo sapiens
  source 1..17: mol_type = unassigned RNA
tggagagaaaggcagta

SEQ ID NO: 32 - RNA, 20 nt, Homo sapiens
  source 1..20: mol_type = unassigned RNA
agccccctggccccaaaccc

SEQ ID NO: 33 - RNA, 18 nt, Homo sapiens
  source 1..18: mol_type = unassigned RNA
ttgcacttgtctcagtga

SEQ ID NO: 34 - RNA, 94 nt, Homo sapiens
  source 1..94: mol_type = unassigned RNA
tggtttgcgactctgaaaactagaaggtttatgactgggcatttctcacccaatgcccaatattgaactttctagttgtcagagtcattaaccc

SEQ ID NO: 35 - RNA, 70 nt, Homo sapiens
  source 1..70: mol_type = unassigned RNA
ttcctgcagattgtttcttttgccgtgcaagtttaagtttttgcacggcaaaagaaacaatccagagggt

SEQ ID NO: 36 - RNA, 73 nt, Homo sapiens
  source 1..73: mol_type = unassigned RNA
actgtccttcagccagagctggctgaagggcagaagggaactgtccttcagccagagctggctgaagggcaga

SEQ ID NO: 37 - RNA, 22 nt, Homo sapiens
  source 1..22: mol_type = unassigned RNA
ttttgcaatatgttcctgaata

SEQ ID NO: 38 - RNA, 21 nt, Homo sapiens
  source 1..21: mol_type = unassigned RNA
gcagtccatgggcatatacac

SEQ ID NO: 39 - RNA, 81 nt, Homo sapiens
  source 1..81: mol_type = unassigned RNA
ggctgcttctcgcctctgtccagctgtgtggccttggacaagcctcttggttacacagctggacagaggcacgaaacagcc

SEQ ID NO: 40 - RNA, 26 nt, Homo sapiens
  source 1..26: mol_type = unassigned RNA
cccagggcttggagtggggcaaggtt

SEQ ID NO: 41 - RNA, 22 nt, Homo sapiens
  source 1..22: mol_type = unassigned RNA
agcaaggcggcatctctctgat

SEQ ID NO: 42 - RNA, 22 nt, Homo sapiens
  source 1..22: mol_type = unassigned RNA
tgcggggacaggccagggcatc

SEQ ID NO: 43 - RNA, 22 nt, Homo sapiens
  source 1..22: mol_type = unassigned RNA
tctgccatcctccctcccctac

SEQ ID NO: 44 - RNA, 21 nt, Homo sapiens
  source 1..21: mol_type = unassigned RNA
agcagacttgacctacaatta

SEQ ID NO: 45 - RNA, 80 nt, Homo sapiens
  source 1..80: mol_type = unassigned RNA
aatgaaggattacggaccagctaagggaggcattaggatccttattcttgcctcccttagttggtccctaatccttcgtt

SEQ ID NO: 46 - RNA, 18 nt, Homo sapiens
  source 1..18: mol_type = unassigned RNA
tggatatgatgactgaaa

SEQ ID NO: 47 - RNA, 21 nt, Homo sapiens
  source 1..21: mol_type = unassigned RNA
gacagagtgccacttactgaa

SEQ ID NO: 48 - RNA, 22 nt, Homo sapiens
  source 1..22: mol_type = unassigned RNA
atgcacctgggcaaggattctg

SEQ ID NO: 49 - RNA, 22 nt, Homo sapiens
  source 1..22: mol_type = unassigned RNA
tgattggtacgtctgtgggtag

SEQ ID NO: 50 - RNA, 22 nt, Homo sapiens
  source 1..22: mol_type = unassigned RNA
tactcaggagagtggcaatcac

SEQ ID NO: 51 - RNA, 21 nt, Homo sapiens
  source 1..21: mol_type = unassigned RNA
ctccagagggaagtactttct

SEQ ID NO: 52 - RNA, 90 nt, Homo sapiens
  source 1..90: mol_type = unassigned RNA
tcccatgctgtgaccctctagaggaagcactttctgtttgttgtctgagaaaaaacaaagtgcttccctttagagtgttaccgtttggga

SEQ ID NO: 53 - RNA, 88 nt, Homo sapiens
  source 1..88: mol_type = unassigned RNA
tcccatgctgtgaccctctagaggaagcactttctgtttgttgtctgagaaaaaacaaagtgcttccctttagagttactgtttggga

SEQ ID NO: 54 - RNA, 91 nt, Homo sapiens
  source 1..91: mol_type = unassigned RNA
cgacttgctttctctcctccatgccttgagtgtaggaccgttggcatcttaattaccctcccacacccaaggcttgcagaagagcgagcct

SEQ ID NO: 55 - RNA, 25 nt, Homo sapiens
  source 1..25: mol_type = unassigned RNA
aaaaaccacaattacttttgcacca

SEQ ID NO: 56 - RNA, 22 nt, Homo sapiens
  source 1..22: mol_type = unassigned RNA
caaaaaccggcaattacttttg

SEQ ID NO: 57 - RNA, 21 nt, Homo sapiens
  source 1..21: mol_type = unassigned RNA
caaaaactgcaattactttca

SEQ ID NO: 58 - RNA, 21 nt, Homo sapiens
  source 1..21: mol_type = unassigned RNA
aaaggtaattgtggtttctgc

SEQ ID NO: 59 - RNA, 21 nt, Homo sapiens
  source 1..21: mol_type = unassigned RNA
taaaaactgcaattactttta

SEQ ID NO: 60 - RNA, 19 nt, Homo sapiens
  source 1..19: mol_type = unassigned RNA
aaaaaccacaattactttt

SEQ ID NO: 61 - RNA, 54 nt, Homo sapiens
  source 1..54: mol_type = unassigned RNA
aaaagtaattgcggtttttgctattggttttaatggcagttacttttgcaccag

SEQ ID NO: 62 - RNA, 22 nt, Homo sapiens
  source 1..22: mol_type = unassigned RNA
aaaagtacttgcggattttgct

SEQ ID NO: 63 - RNA, 25 nt, Homo sapiens
  source 1..25: mol_type = unassigned RNA
aaaaaccacaattacttttgcacca

SEQ ID NO: 64 - RNA, 20 nt, Homo sapiens
  source 1..20: mol_type = unassigned RNA
taaaaactgcaattactttc

SEQ ID NO: 65 - RNA, 110 nt, Homo sapiens
  source 1..110: mol_type = unassigned RNA
gcctaaactattaggttggtgcaaaagtaatcactgtttttgccattactctcagtggcaaaaaccgtgattacttttgcaccaacctagtaacaccttcactgtggggg

SEQ ID NO: 66 - RNA, 97 nt, Homo sapiens
  source 1..97: mol_type = unassigned RNA
cttcatccaccagtcctccaggaacatcaaggatcttaaactttgccagagctacaaaggcaaagtttaagatccttgaagttcctgggggaaccat

SEQ ID NO: 67 - RNA, 22 nt, Homo sapiens
  source 1..22: mol_type = unassigned RNA
cacacactgcaattacttttgc

SEQ ID NO: 68 - RNA, 21 nt, Homo sapiens
  source 1..21: mol_type = unassigned RNA
aaactactgaaaatcaaagat

SEQ ID NO: 69 - RNA, 95 nt, Homo sapiens
  source 1..95: mol_type = unassigned RNA
gacaccacatgctcctccaggcctgcctgccctccaggtcatgttccagtgtcccacagatgcagcaccacggcccaggcggcattggtgtcacc

SEQ ID NO: 70 - RNA, 97 nt, Homo sapiens
  source 1..97: mol_type = unassigned RNA
ccaccacggtgctggcaccagggcctctgccccgtaggacaccgaggcttatgaataggagcagtgccggccaaggcgccggcaccatcttggtgat

SEQ ID NO: 71 - RNA, 22 nt, Homo sapiens
  source 1..22: mol_type = unassigned RNA
tgggaaagagaaagaacaagta

SEQ ID NO: 72 - RNA, 22 nt, Homo sapiens
  source 1..22: mol_type = unassigned RNA
tcgggcctggggttgggggagc

SEQ ID NO: 73 - RNA, 59 nt, Homo sapiens
  source 1..59: mol_type = unassigned RNA
cgggctctgggtgcagtgggggttcccacgccgcggcaaccaccactgtctctccccag

SEQ ID NO: 74 - RNA, 25 nt, Homo sapiens
  source 1..25: mol_type = unassigned RNA
taggggtgggggaattcaggggtgt

SEQ ID NO: 75 - RNA, 20 nt, Homo sapiens
  source 1..20: mol_type = unassigned RNA
tccccaacccctgcccgcag

SEQ ID NO: 76 - RNA, 61 nt, Homo sapiens
  source 1..61: mol_type = unassigned RNA
gtgcggaacgctggccggggcgggaggggaagggacgcccggccggaacgccgcactcacg

SEQ ID NO: 77 - RNA, 21 nt, Homo sapiens
  source 1..21: mol_type = unassigned RNA
acaccctctttccctaccgcc

SEQ ID NO: 78 - RNA, 22 nt, Homo sapiens
  source 1..22: mol_type = unassigned RNA
cagagggaatacagagggcaat

SEQ ID NO: 79 - RNA, 67 nt, Homo sapiens
  source 1..67: mol_type = unassigned RNA
tggagctgtgtgcagggccagcgcggagcccgagcagccgcggtgaagcgcctgtgctctgccgaga

SEQ ID NO: 80 - RNA, 20 nt, Homo sapiens
  source 1..20: mol_type = unassigned RNA
tgtgaccctagaataattac

SEQ ID NO: 81 - RNA, 22 nt, Homo sapiens
  source 1..22: mol_type = unassigned RNA
cgggactgtagagggcatgagc

SEQ ID NO: 82 - RNA, 80 nt, Homo sapiens
  source 1..80: mol_type = unassigned RNA
ccttgctgatggcagatgtcggatctgcctcgcttatacgtgcccttgctgatggcagatgtcgggtctgcctcgcttat

SEQ ID NO: 83 - RNA, 82 nt, Homo sapiens
  source 1..82: mol_type = unassigned RNA
aaggagcactcactccaatttccctggactgggggcaggctgccacctcctggggacaggggattggggcaggatgttccag

SEQ ID NO: 84 - RNA, 77 nt, Homo sapiens
  source 1..77: mol_type = unassigned RNA
tgcaatgccctactcagaaaggtgccatttatgtagattttatgtcactggctcctttctgggtagagcaaggctca

SEQ ID NO: 85 - RNA, 22 nt, Homo sapiens
  source 1..22: mol_type = unassigned RNA
acagtagagggaggaatcgcag

SEQ ID NO: 86 - RNA, 20 nt, Homo sapiens
  source 1..20: mol_type = unassigned RNA
gtgagtcagggtggggctgg

SEQ ID NO: 87 - RNA, 69 nt, Homo sapiens
  source 1..69: mol_type = unassigned RNA
agaggcttgggccgccgagctggacccggaccggttttgggtactgtactgggggcagggcagagaggg

SEQ ID NO: 88 - RNA, 106 nt, Homo sapiens
  source 1..106: mol_type = unassigned RNA
gtgctcgcttcggcagcacatatactaaaattggaacgatacagagaagattagcatggcccctgcgcaaggatgacacgcaaattcgtgaagcgttccatatttt

(19) United States
(12) Patent Application Publication
(10) Pub. No.: US 2026/0092326 A1
(43) Pub. Date: Apr. 2, 2026

(54) CIRCULATING SERUM MICRORNA BIOMARKERS AND METHODS FOR ALZHEIMER'S DISEASE DIAGNOSIS

(71) Applicant: ST. JOHN’S UNIVERSITY, Queens, NY (US)

(72) Inventors: Simon Geir Möller, Queens, NY (US); Ketan Shirish Patil, Queens, NY (US); Guido Werner Alves, Stavanger (NO)

(21) Appl. No.: 19/280,334

(22) Filed: Jul. 25, 2025

Related U.S. Application Data
(62) Division of application No. 18/182,868, filed on Mar. 13, 2023, now Pat. No. 12,398,427.
Application No. 18/182,868 is a division of application No. 16/623,937, now Pat. No. 11,634,775.
(60) Provisional application No. 62/521,768, filed on Jun. 19, 2017.

(51) Int. Cl. C12Q 1/6883

(52) U.S. Cl. CPC C12Q 1/6883; C12Q 2600/158; C12Q 2600/178; G01N 2800/2821

(57) ABSTRACT

Biomarkers and methods for identifying, verifying and confirming circulating serum-based microRNAs. The microRNAs (PARKmiRs) can be used to differentiate patient's suffering from Alzheimer's disease (AD) from non-AD patients.

This application is a divisional of U.S. patent application Ser. No. 18/182,868 filed Mar. 13, 2023 which is a divisional of U.S. patent application Ser. No. 16/623,937 filed Dec. 18, 2019 issued as U.S. Pat. No. 11,634,775 on Apr. 25, 2023, which claims benefit of PCT Application No. PCT/US2018/036377 filed Jun. 7, 2018, which in turn claims benefit of U.S. Provisional Application No. 62/521,768 filed Jun. 19, 2017.

The 80,360 bytes Sequence Listing XML file named 140415_614515_SL.xml, created on Jun. 8, 2023 is hereby incorporated by reference. The sequence data presented in the Sequence Listing XML file may be found in Table 1 at specification paragraph [0027], and so that Sequence Listing XML file contains no new matter.

### BACKGROUND OF THE INVENTION

### 1. Field of the Invention

The present invention generally relates to serum-based microRNAs and methods for differentiating patients suffering from Alzheimer's disease, as well as assisting clinicians to determine treatment protocols for such patients.

### 2. Brief Description of the Background Art

Alzheimer's disease (AD), the most common neurodegenerative disease, is characterized by loss of memory and other cognitive abilities of an individual with treatment available for only symptomatic relief. Alzheimer's is a progressive disease, which advances with increasingly severe symptoms including mood and behavior changes: difficulty speaking, swallowing and walking: disorientation and more serious memory loss. The drug combinations in use are only palliative but cannot reverse the process of neuronal cell death. There are neither any objective tests nor any established biomarkers for the diagnosis of AD. Further, the heterogeneity, subtypes and the progression of the disease makes it even complex to develop specific therapeutic candidates. Thus it is imperative to diagnose disease at the early stage to increase the efficacy of therapeutic agents.

AD and AD related dementia currently affects about 44 million people world-wide. Effective management of a patient with AD is possible in the initial years of treatment, after which time a series of often debilitating complications occur. Current treatment for AD includes multi-drug regiment including cholinesterase inhibitors, Antidepressants, Anxiolytics, Antipsychotic medications, and sedatives to treat a specific symptom. There are many new drugs being developed that can alter the disease process itself by targeting AD-related proteins and processes including beta-amyloid, beta-secretase, Tau-protein, inflammation, and the 5HT6 receptor amongst others.

In the brain, neurons connect and communicate at synapses, where tiny bursts of chemicals called neurotransmitters carry information from one cell to another. Neurons are the chief cells destroyed by Alzheimer's disease. Accordingly, Alzheimer's disease destroys synapses and kills neurons, damaging and eventually destroying the brain's communication network.

Current FDA-approved Alzheimer's drugs support this communication process through two different mechanisms:
1) Cholinesterase inhibitors work by slowing down the process that breaks down a key neurotransmitter. Specifically, cholinesterase inhibitors boost levels of cell-to-cell communication by providing the neurotransmitter acetylcholine that is depleted in the brain by Alzheimer's disease. Donepezil, galantamine and rivastigmine are cholinesterase inhibitors.
2) Memantine is an NMDA (N-methyl-D)-aspartate) receptor antagonist and works by regulating the activity of glutamate, a neurotransmitter in the brain. Attachment of glutamate to cell surface NMDA receptors permits calcium to enter the cell. This process is important for cell signaling, as well as learning and memory. In Alzheimer's disease, excess glutamate can be released from damaged cells, leading to chronic overexposure to calcium, which can speed up cell damage. Memantine helps prevent this destructive chain of events by partially blocking the NMDA receptors.

Although the effectiveness of cholinesterase inhibitors and memantine varies widely across the population, it is imperative to diagnose individuals with AD at an early stage to increase the efficacy of therapeutic agents. However, there are neither any objective tests nor established biomarkers for diagnosing AD. Moreover, the heterogeneity, subtypes and progression of the disease make it difficult to develop specific therapeutic candidates.

MicroRNAs (“miRNAs) are a class of non-coding RNAs that play key roles in the regulation of gene expression. miRNAs act at the post-transcriptional level and fine-tune the expression of as much as 30% of all mammalian protein-encoding genes. Mature miRNAs are short, single-stranded RNA molecules approximately 22 nucleotides in length. miRNAs may be encoded by multiple loci, and may be organized in tandemly co-transcribed clusters. miRNA genes are transcribed by RNA polymerase II as large primary transcripts (pri-microRNA) that are processed by a protein complex containing the RNase III enzyme Drosha, DGCR8 and other cofactors, to form an approximately 70 nucleotide precursor microRNA (pre-miRNA). (Cathew R W, Cell, 2009; Kim V N, Nat Rev Mol Cel Biol, 2009; Siomi H, Mol Cel, 2010; Bartel D P, Cell, 2004; Lee Y, Nature 2003; Han J, Genes Dev, 2004.) Pre-miRNA is transported to the cytoplasm by Exportin-5 where it is processed by DICER, a second RNase III enzyme, together with TRBP, PACT and Ago2 in the RNA Induced Silencing Complex resulting in miRNA duplexes (Kim V N, Nat Rev Mol Cel Biol, 2009: Gregory R I, Nature 2004; MAcRae I J, PNAS, 2008). The guide strands of miRNA duplexes separate and associate with Ago 2 for incorporation into a ribonuclear particle to form the RNA-induced silencing complex RISC that mediates gene silencing. The mechanisms of miRNA range from direct degradation or silencing of mRNA and repression of translation to post-transcriptional upregulations. (MacRae I J, PNAS, 2008.)

The presence of miRNAs has been reported in body fluids including blood, cerebrospinal fluid (CSF), plasma, serum and saliva at detectable levels. The tissue-specificity of miRNAs suggests their vital and integral role in various physiological processes. The tissue-enrichment promises a new but less explored role as diagnostic biomarker and potential therapeutic target. Circulating miRNAs are understood to originate from passive leakage from damaged tissue as a result of cell lysis or apoptosis, active transport from cells via microvesicles, such as exosomes, or bound within RISC protein complexes (Etheridge et al, 2011). Exosome and osmotic pump-mediated delivery of small RNA molecules to the brain and CNS, respectively, provides a solution to overcoming the limitations of miRNA-based therapies (Alvarez-Erviti et al., 2011: Koval et al, 2013, Hum. Mol. Gen). miRNA has been demonstrated to be exceptionally stable and thus present as powerful candidates to be potential biomarkers (Chen et al, 2008: Grasso, 2014).

### SUMMARY OF THE INVENTION

It is an object of the present invention to identify miRNAs relevant to patients suffering from Alzheimer's disease.

It is another object of the present invention to provide methods for determining patients suffering from Alzheimer's disease.

These objects and others are achieved by the present invention, which provides miRNA biomarkers that may be used singly, in pairs or in combination to determine patients suffering from Alzheimer's disease.

### BRIEF DESCRIPTION OF THE DRAWINGS

FIG. 1 shows the mean fold change of three PARKmiRNAs between AD patients and healthy controls.

FIG. 2 shows the mean fold change of three combinations of PARKmiRNAs between AD patients and healthy controls.

### DETAILED DESCRIPTION OF THE INVENTION

We performed microarray analysis (discovery phase from the Norwegian ParkWest study), confirmation by qRT-PCR (same samples from discovery phase), verification by qRT-PCR (large sample set from the Norwegian Parkwest study) and validation by qRT-PCR (independent cohort from the Swedish NYPUM study) on control and PD serum samples at baseline as described in the PD diagnostic patent. All this data was generated and discussed in U.S. Application No. 62/291,619 filed Feb. 5, 2016 and International Application No. PCT/US2017/016412 filed Feb. 3, 2017, the disclosures of which are hereby incorporated herein by reference.

During data collection for the diagnostic PD miRNA project we also tested the candidate miRNAs (PARKmiRs) for specificity using 45 serum samples from newly diagnosed AD patients from the DemVest study representing the same region in Norway as for the PD population in the Parkwest study. The inventors expected that the PARKmiRs would show the same abundance levels as in control serum samples, which would verify specificity of the PARKmiRs to PD. Unexpectedly the PARKmiRs showed a significant decrease in levels in the AD serum samples as compared to control serum samples. To ensure that the AD serum samples and the techniques used were valid we tested whether miR-445-3p and control small RNA (U6) changed in abundance. In control serum, PD serum and AD serum both miRNAs remained unchanged in abundance validating our findings.

### Methods

### Serum Samples Handling and Classification

All patients and controls participated in the Norwegian ParkWest study and the Dementia Study of Western Norway (DemVest study) which are ongoing prospective population-based longitudinal cohort studies investigating the incidence, neurobiology and prognosis of PD and dementia/AD, respectively. The Norwegian Park West study is a prospective longitudinal multicenter cohort study of patients with incident Parkinson's disease (PD) from Western and Southern Norway. Between Nov. 1, 2004 and 31 Aug. 2006 it was endeavored to recruit all new cases of Parkinson Disease within the study area. Since the start of the study 212 of 265 (80%) of these patients and their age-/sex-matched control group have been followed. The Dementia Study of Western Norway is a prospective longitudinal multicenter cohort study of patients with a first-time dementia diagnosis (Mini Mental State Examination (MMSE) score >15). Patient recruitment started in 2005 and patients were followed annually. Patients with acute delirium or confusion, terminal illness, or current or previous bipolar disorder or psychotic disorder, or who were recently diagnosed with a major somatic illness, were excluded from the study.

All possible efforts were undertaken to establish an unselected and population-representative cohort of patients with AD. Patients were included if they had provided serum at study entry and fulfilled diagnostic criteria for AD according to the National Institute of Neurological and Communicative Diseases and Stroke/Alzheimer's Disease and Related Disorders Association (NINCDS/ARDRA) criteria at latest follow-up. Control subjects were recruited from multiple sources, including friends, spouses, and public organizations for elderly and were included in this study if they had provided serum.

In this study of possible biomarkers for AD we applied a two-stage procedure. For the first discovery phase serum from 16 patients and 8 controls were selected at random. The remaining 45 patients with AD and 182 controls that were eligible for this study were selected for verification purposes.

Serum samples were collected at the same day as the clinical examinations and then stored frozen at −70 degrees Celsius until transported to the facilities in New York on dry ice.

### Example 1: Analyses of Differentially Expressed Human miRNA by qPCR

RNA Isolation from Serum Samples and QC

After thawing on ice, twenty-four (eight control, sixteen PD samples) serum samples were spun down for 5 mins at 3000×g to remove debris. The supernatant was used to perform small RNA isolation using miRCURY RNA Isolation Kit—Biofluids (Exiqon, MA). Before RNA Isolation, the lysis buffer was spiked with 0.267 fmol/ul of spike-in control cel-miR-39-3p (Qiagen, CA). The remaining part of the RNA isolation was performed following the manufacturer's protocol and the isolated RNA was quantified on a Nanodrop 2000 (Thermo Scientific, MA). The RNA was used for running Affymetrix v4 microRNA microarray chips and for subsequent cDNA synthesis and qPCR. RNA from 256 serum samples (190 control, 16 PD from ParkWest project 45 AD from the DemVest project) was isolated as described above, they were not quantified by Nanodrop, but the qPCR data resulting from these samples were normalized by a reference small RNA scaRNA17.

miRNA Microarray and Data Analysis

The isolated RNA from twenty-four patient serum samples were quantified and subjected to Affymetrix GeneChip® miRNA 4.0 Array by the Yale Center for Genome Analysis. The normalized .CEL files obtained from Affymetrix Expression Console software were imported into Partek Genomics Suite version 6.6 Copyright @ 2012 (Partek, MO) for analysis. The ‘microRNA Expression Workflow’ was employed to detect differentially expressed miRNAs employing ANOVA resulting in lists of miRNAs significantly (p<0.05) expressed between control versus PD cohorts. The miRNAs detected were used for further qPCR verification.

### Quantitative Polymerase Chain Reaction

cDNA for miRNA specific qPCR was synthesized using qScript™ microRNA cDNA Synthesis kit (Quanta Biosciences, MD) following manufacturer's protocol and subsequent qPCRs were performed using miRNA specific forward primers (Table #) and PerfeCTa® Universal PCR primer (Quanta Biosciences, MD). scaRNA17 and U6 were used reference small RNAs for normalizing qPCR Cq values whereas cel-miR-39-3p was used as spike-in control. PerfeCTa® SYBR® GREEN SuperMix for IQ™ (Quanta Biosciences, MD) was used for all qPCRs in a MyiQ™ Single color Real-Time PCR Detection System (Bio-Rad, CA). Standard curve for cel-miR-39-3p was analyzed in MS Excel with R²=0.97882 and PCR efficiency 92.96%. No Template Control (NTC) was implied wherever needed.

### Data Analysis Based on PD Model

The discriminative ability of miRNAs with regard to PD diagnosis was assessed from ROC analysis using IBM SPSS Statistics, version 21: for combinations of miRNAs the test variable was the predicted probability from logistic regression with PD diagnosis (yes/no) as outcome. To minimize the influence of outlying values on the fit, logistic regression was performed with log transformed miRNA values.

Differentially expressed human miRNAs in PD patients' serum samples from The Norwegian Park West study were determined employing miRNA microarray. Provided below are the miRNAs with >1.2 fold differential expression.

85 Differentially Expressed Human Pre- and Mature miRNAs with >1.2 Fold Change
hsa-miR-548ac, hsa-miR-335-5p, hsa-miR-548x-3p, hsa-miR-520g, hsa-miR-520h, hsa-miR-548ae, hsa-miR-3910-1, hsa-miR-4708-3p, hsa-miR-16-2-3p, hsa-miR-603, hsa-miR-3613-3p, hsa-miR-4797-5p, hsa-miR-548aj-3p, hsa-miR-450b-5p, hsa-miR-548ap-3p, hsa-miR-1184, hsa-miR-2277-5p, hsa-miR-1323, hsa-miR-548aa, hsa-miR-548t-3p, hsa-miR-221-5p, hsa-miR-190a-3p, hsa-miR-6873-5p, hsa-miR-155-3p, hsa-miR-510-5p, hsa-miR-4313, hsa-miR-3616, hsa-miR-8075, hsa-miR-4306, hsa-miR-6776, hsa-miR-6075, hsa-miR-8052, hsa-miR-532, hsa-miR-4791, hsa-miR-320b-1, hsa-miR-548y, hsa-miR-7973, hsa-miR-3136-5p, hsa-miR-606, hsa-miR-500a-3p, hsa-miR-4788, hsa-miR-4769-3p, hsa-miR-299-5p, hsa-miR-4431, hsa-miR-6749-5p, hsa-miR-138-2-3p, hsa-miR-1289-2, hsa-miR-548au, hsa-miR-6850, hsa-miR-561, hsa-miR-34b-5p, hsa-miR-3934-5p, hsa-miR-6739-5p, hsa-miR-4325, hsa-miR-4672, hsa-miR-215-5p, hsa-miR-4685-5p, hsa-miR-3160-1, hsa-miR-3160-2, hsa-miR-6793-5p, hsa-miR-8089, hsa-miR-6081, hsa-miR-892b, hsa-miR-936, hsa-miR-548ag, hsa-miR-345, hsa-miR-548k, hsa-miR-3188, hsa-miR-181b-5p, hsa-let-7e, hsa-miR-4487, hsa-miR-509-3p, hsa-miR-3689a-3p, hsa-miR-4771, hsa-miR-520a-5p, hsa-miR-3150b, hsa-miR-6782-5p, hsa-miR-937-5p, hsa-miR-455-3p, hsa-miR-6865-3p, hsa-miR-4749-5p, hsa-miR-378b, hsa-miR-7706, hsa-miR-4445 and hsa-miR-2355-5p.
   57 Differentially Expressed Mature miRNAs with >1.2 Fold Change
hsa-miR-548ac, hsa-miR-335-5p, hsa-miR-548x-3p, hsa-miR-548ae, hsa-miR-4708-3p, hsa-miR-16-2-3p, hsa-miR-603, hsa-miR-3613-3p, hsa-miR-4797-5p, hsa-miR-548aj-3p, hsa-miR-450b-5p, hsa-miR-548ap-3p, hsa-miR-1184, hsa-miR-2277-5p, hsa-miR-1323, hsa-miR-548aa, hsa-miR-548t-3p, hsa-miR-221-5p, hsa-miR-190a-3p, hsa-miR-6873-5p, hsa-miR-155-3p, hsa-miR-510-5p, hsa-miR-4313, hsa-miR-4306, hsa-miR-8052, hsa-miR-4791, hsa-miR-7973, hsa-miR-3136-5p, hsa-miR-606, hsa-miR-500a-3p, hsa-miR-4769-3p, hsa-miR-299-5p, hsa-miR-6749-5p, hsa-miR-138-2-3p, hsa-miR-34b-5p, hsa-miR-3934-5p, hsa-miR-6739-5p, hsa-miR-4325, hsa-miR-215-5p, hsa-miR-4685-5p, hsa-miR-6793-5p, hsa-miR-936, hsa-miR-548ag, hsa-miR-548k, hsa-miR-181b-5p, hsa-let-7e, hsa-miR-509-3p, hsa-miR-3689a-3p, hsa-miR-4771, hsa-miR-520a-5p, hsa-miR-6782-5p, hsa-miR-937-5p, hsa-miR-455-3p, hsa-miR-6865-3p, hsa-miR-4749-5p, hsa-miR-378b and hsa-miR-2355-5p.
   28 Differentially Expressed Premature miRNAs with >1.2 Fold Change
hsa-miR-520g, hsa-miR-520h, hsa-miR-3910-1, hsa-miR-3616, hsa-miR-8075, hsa-miR-6776, hsa-miR-6075, hsa-miR-532, hsa-miR-320b-1, hsa-miR-548y, hsa-miR-4788, hsa-miR-4431, hsa-miR-1289-2, hsa-miR-548au, hsa-miR-6850, hsa-miR-561, hsa-miR-4672, hsa-miR-3160-1, hsa-miR-3160-2, hsa-miR-8089, hsa-miR-6081, hsa-miR-892b, hsa-miR-345, hsa-miR-3188, hsa-miR-4487, hsa-miR-3150b, hsa-miR-7706 and hsa-miR-4445.

These differentially expressed miRNA sequences are illustrated below in Table 1, along with the reference/house-keeping small RNAs cel-miR-39-3p, U6 and ScaRNA17 used as controls. Cel-miR-39-3p is a spike-in control that demonstrates the stability of the RNA samples. U6 and ScaRNA17 are used as internal controls to normalize the readings of the rest of the miRNAs or candidate miRNAs.

TABLE 1

| microRNA/ | |
|---|---|
| small RNA name | microRNA Sequence |
| cel-miR-39-3p | UCACCGGGUGUAAAUCAGCUUG (SEQ ID NO: 1) |
| hsa-let-7e | CCCGGGCUGAGGUAGGAGGUUGUAUAGUUGAGGAGGACACCC |
| | AAGGAGAUCACUAUACGGCCUCCUAGCUUUCCCCAGG |
| | (SEQ ID NO: 2) |
| hsa-miR-1184 | CCUGCAGCGACUUGAUGGCUUCC (SEQ ID NO: 3) |
| hsa-miR-1289-2 | CCACGGUCCUAGUUAAAAAGGCACAUUCCUAGACCCUGCCUC |
| | AGAACUACUGAACAGAGUCACUGGGUGUGGAGUCCAGGAAUC |
| | UGCAUUUUUACCCCUAUCGCCCCCGCC (SEQ ID NO: 4) |
| hsa-miR-1323 | UCAAAACUGAGGGGCAUUUUCU (SEQ ID NO: 5) |
| hsa-miR-138-2-3p | GCUAUUUCACGACACCAGGGUU (SEQ ID NO: 6) |
| hsa-miR-155-3p | CUCCUACAUAUUAGCAUUAACA (SEQ ID NO: 7) |
| hsa-miR-16-2-3p | CCAAUAUUACUGUGCUGCUUUA (SEQ ID NO: 8) |
| hsa-miR-181b-5p | AACAUUCAUUGCUGUCGGUGGGU (SEQ ID NO: 9) |
| hsa-miR-190a-3p | CUAUAUAUCAAACAUAUUCCU (SEQ ID NO: 10) |
| hsa-miR-215-5p | AUGACCUAUGAAUUGACAGAC (SEQ ID NO: 11) |
| hsa-miR-221-5p | ACCUGGCAUACAAUGUAGAUUU (SEQ ID NO: 12) |
| hsa-miR-2277-5p | AGCGCGGGCUGAGCGCUGCCAGUC (SEQ ID NO: 13) |
| hsa-miR-2355-5p | AUCCCCAGAUACAAUGGACAA (SEQ ID NO: 14) |
| hsa-miR-299-5p | UGGUUUACCGUCCCACAUACAU (SEQ ID NO: 15) |
| hsa-miR-3136-5p | CUGACUGAAUAGGUAGGGUCAUU (SEQ ID NO: 16) |
| hsa-miR-3150b | GAGGGAAAGCAGGCCAACCUCGAGGAUCUCCCCAGCCUUGGC |
| | GUUCAGGUGCUGAGGAGAUCGUCGAGGUUGGCCUGCUUCCCC |
| | UC (SEQ ID NO: 17) |
| hsa-miR-3160-1 | GGACCUGCCCUGGGCUUUCUAGUCUCAGCUCUCCUCCAGCUC |
| | AGCUGGUCAGGAGAGCUGAGACUAGAAAGCCCAGGGCAGGUU |
| | C (SEQ ID NO: 18) |
| hsa-miR-3160-2 | ACCUGCCCUGGGCUUUCUAGUCUCAGCUCUCCUGACCAGCUG |
| | AGCUGGAGGAGAGCUGAGACUAGAAAGCCCAGGGCAGGU |
| | (SEQ ID NO: 19) |
| hsa-miR-3188 | GGCGCCUCCUGCUCUGCUGUGCCGCCAGGGCCUCCCCUAGCGC |
| | GCCUUCUGGAGAGGCUUUGUGCGGAUACGGGGCUGGAGGCCU |
| | (SEQ ID NO: 20) |
| hsa-miR-320b-1 | AAUUAAUCCCUCUCUUUCUAGUUCUUCCUAGAGUGAGGAAAA |
| | GCUGGGUUGAGAGGGCAAACAAAUUAACUAAUUAAUU |
| | (SEQ ID NO: 21) |
| hsa-miR-335-5p | UCAAGAGCAAUAACGAAAAAUGU (SEQ ID NO: 22) |
| hsa-miR-345 | ACCCAAACCCUAGGUCUGCUGACUCCUAGUCCAGGGCUCGUG |
| | AUGGCUGGUGGGCCCUGAACGAGGGGUCUGGAGGCCUGGGUU |
| | UGAAUAUCGACAGC (SEQ ID NO: 23) |
| hsa-miR-34b-5p | UAGGCAGUGUCAUUAGCUGAUUG (SEQ ID NO: 24) |
| hsa-miR-3613-3p | ACAAAAAAAAAAGCCCAACCCUUC (SEQ ID NO: 25) |
| hsa-miR-3616 | UGUCACUCCGCCAGCAUCAUGAAGUGCACUCAUGAUAUGUUU |
| | GCCCCAUCAGCGUGUCACGAGGGCAUUUCAUGAUGCAGGCGG |
| | GGUUGGCA (SEQ ID NO: 26) |
| hsa-miR-3689a-3p | CUGGGAGGUGUGAUAUCGUGGU (SEQ ID NO: 27) |
| hsa-miR-378b | ACUGGACUUGGAGGCAGAA (SEQ ID NO: 28) |
| hsa-miR-3910-1 | CUUUUGCUGUCAGUUUUUCUGUUGCUUGUCUUGGUUUUAUGC |
| | CUUUUAUAUCAAGGCACAUAAAAGGCAUAAAACCAAGACAAG |
| | CAACAAAAAAAGGAUUGAUCACAGAAG (SEQ ID NO: 29) |
| hsa-miR-3934-5p | UCAGGUGUGGAAACUGAGGCAG (SEQ ID NO: 30) |
| hsa-miR-4306 | UGGAGAGAAAGGCAGUA (SEQ ID NO: 31) |
| hsa-miR-4313 | AGCCCCCUGGCCCCAAACCC (SEQ ID NO: 32) |
| hsa-miR-4325 | UUGCACUUGUCUCAGUGA (SEQ ID NO: 33) |
| hsa-miR-4431 | UGGUUUGCGACUCUGAAAACUAGAAGGUUUAUGACUGGGCA |
| | UUUCUCACCCAAUGCCCAAUAUUGAACUUUCUAGUUGUCAGA |
| | GUCAUUAACCC (SEQ ID NO: 34) |
| hsa-miR-4445 | UUCCUGCAGAUUGUUUCUUUUGCCGUGCAAGUUUAAGUUUUU |
| | GCACGGCAAAAGAAACAAUCCAGAGGGU (SEQ ID NO: 35) |
| hsa-miR-4487 | ACUGUCCUUCAGCCAGAGCUGGCUGAAGGGCAGAAGGGAACU |
| | GUCCUUCAGCCAGAGCUGGCUGAAGGGCAGA |
| | (SEQ ID NO: 36) |
| hsa-miR-450b-5p | UUUUGCAAUAUGUUCCUGAAUA (SEQ ID NO: 37) |
| hsa-miR-455-3p | GCAGUCCAUGGGCAUAUACAC (SEQ ID NO: 38) |
| hsa-miR-4672 | GGCUGCUUCUCGCCUCUGUCCAGCUGUGUGGCCUUGGACAAG |
| | CCUCUUGGUUACACAGCUGGACAGAGGCACGAAACAGCC |
| | (SEQ ID NO: 39) |
| hsa-miR-4685-5p | CCCAGGGCUUGGAGUGGGGCAAGGUU (SEQ ID NO: 40) |
| hsa-miR-4708-3p | AGCAAGGCGGCAUCUCUCUGAU (SEQ ID NO: 41) |
| hsa-miR-4749-5p | UGCGGGGACAGGCCAGGGCAUC (SEQ ID NO: 42) |
| hsa-miR-4769-3p | UCUGCCAUCCUCCCUCCCCUAC (SEQ ID NO: 43) |
| hsa-miR-4771 | AGCAGACUUGACCUACAAUUA (SEQ ID NO: 44) |
| hsa-miR-4788 | AAUGAAGGAUUACGGACCAGCUAAGGGAGGCAUUAGGAUCCU |
| | UAUUCUUGCCUCCCUUAGUUGGUCCCUAAUCCUUCGUU |
| | (SEQ ID NO: 45) |
| hsa-miR-4791 | UGGAUAUGAUGACUGAAA (SEQ ID NO: 46) |
| hsa-miR-4797-5p | GACAGAGUGCCACUUACUGAA (SEQ ID NO: 47) |
| hsa-miR-500a-3p | AUGCACCUGGGCAAGGAUUCUG (SEQ ID NO: 48) |
| hsa-miR-509-3p | UGAUUGGUACGUCUGUGGGUAG (SEQ ID NO: 49) |
| hsa-miR-510-5p | UACUCAGGAGAGUGGCAAUCAC (SEQ ID NO: 50) |
| hsa-miR-520a-5p | CUCCAGAGGGAAGUACUUUCU (SEQ ID NO: 51) |
| hsa-miR-520g | UCCCAUGCUGUGACCCUCUAGAGGAAGCACUUUCUGUUUGUU |
| | GUCUGAGAAAAAACAAAGUGCUUCCCUUUAGAGUGUUACCGU |
| | UUGGGA (SEQ ID NO: 52) |
| hsa-miR-520h | UCCCAUGCUGUGACCCUCUAGAGGAAGCACUUUCUGUUUGUU |
| | GUCUGAGAAAAAACAAAGUGCUUCCCUUUAGAGUUACUGUUU |
| | GGGA (SEQ ID NO: 53) |
| hsa-miR-532 | CGACUUGCUUUCUCUCCUCCAUGCCUUGAGUGUAGGACCGUU |
| | GGCAUCUUAAUUACCCUCCCACACCCAAGGCUUGCAGAAGAG |
| | CGAGCCU (SEQ ID NO: 54) |
| hsa-miR-548aa | AAAAACCACAAUUACUUUUGCACCA (SEQ ID NO: 55) |
| hsa-miR-548ac | CAAAAACCGGCAAUUACUUUUG (SEQ ID NO: 56) |
| hsa-miR-548ae | CAAAAACUGCAAUUACUUUCA (SEQ ID NO: 57) |
| hsa-miR-548ag | AAAGGUAAUUGUGGUUUCUGC (SEQ ID NO: 58) |
| hsa-miR-548aj-3p | UAAAAACUGCAAUUACUUUUA (SEQ ID NO: 59) |
| hsa-miR-548ap-3p | AAAAACCACAAUUACUUUU (SEQ ID NO: 60) |
| hsa-miR-548au | AAAAGUAAUUGCGGUUUUUGCUAUUGGUUUUAAUGGCAGUU |
| | ACUUUUGCACCAG (SEQ ID NO: 61) |
| hsa-miR-548k | AAAAGUACUUGCGGAUUUUGCU (SEQ ID NO: 62) |
| hsa-miR-548t-3p | AAAAACCACAAUUACUUUUGCACCA (SEQ ID NO: 63) |
| hsa-miR-548x-3p | UAAAAACUGCAAUUACUUUC (SEQ ID NO: 64) |
| hsa-miR-548y | GCCUAAACUAUUAGGUUGGUGCAAAAGUAAUCACUGUUUUU |
| | GCCAUUACUCUCAGUGGCAAAAACCGUGAUUACUUUUGCACC |
| | AACCUAGUAACACCUUCACUGUGGGGG (SEQ ID NO: 65) |
| hsa-miR-561 | CUUCAUCCACCAGUCCUCCAGGAACAUCAAGGAUCUUAAACU |
| | UUGCCAGAGCUACAAAGGCAAAGUUUAAGAUCCUUGAAGUUC |
| | CUGGGGGAACCAU (SEQ ID NO: 66) |
| hsa-miR-603 | CACACACUGCAAUUACUUUUGC (SEQ ID NO: 67) |
| hsa-miR-606 | AAACUACUGAAAAUCAAAGAU (SEQ ID NO: 68) |
| hsa-miR-6075 | GACACCACAUGCUCCUCCAGGCCUGCCUGCCCUCCAGGUCAU |
| | GUUCCAGUGUCCCACAGAUGCAGCACCACGGCCCAGGCGGCA |
| | UUGGUGUCACC (SEQ ID NO: 69) |
| hsa-miR-6081 | CCACCACGGUGCUGGCACCAGGGCCUCUGCCCCGUAGGACAC |
| | CGAGGCUUAUGAAUAGGAGCAGUGCCGGCCAAGGCGCCGGCA |
| | CCAUCUUGGUGAU (SEQ ID NO: 70) |
| hsa-miR-6739-5p | UGGGAAAGAGAAAGAACAAGUA (SEQ ID NO: 71) |
| hsa-miR-6749-5p | UCGGGCCUGGGGUUGGGGGAGC (SEQ ID NO: 72) |
| hsa-miR-6776 | CGGGCUCUGGGUGCAGUGGGGGUUCCCACGCCGCGGCAACCA |
| | CCACUGUCUCUCCCCAG (SEQ ID NO: 73) |
| hsa-miR-6782-5p | UAGGGGUGGGGGAAUUCAGGGGUGU (SEQ ID NO: 74) |
| hsa-miR-6793-5p | UCCCCAACCCCUGCCCGCAG (SEQ ID NO: 75) |
| hsa-miR-6850 | GUGCGGAACGCUGGCCGGGGCGGGAGGGGAAGGGACGCCCGG |
| | CCGGAACGCCGCACUCACG (SEQ ID NO: 76) |
| hsa-miR-6865-3p | ACACCCUCUUUCCCUACCGCC (SEQ ID NO: 77) |
| hsa-miR-6873-5p | CAGAGGGAAUACAGAGGGCAAU (SEQ ID NO: 78) |
| hsa-miR-7706 | UGGAGCUGUGUGCAGGGCCAGCGCGGAGCCCGAGCAGCCGCG |
| | GUGAAGCGCCUGUGCUCUGCCGAGA (SEQ ID NO: 79) |
| hsa-miR-7973 | UGUGACCCUAGAAUAAUUAC (SEQ ID NO: 80) |
| hsa-miR-8052 | CGGGACUGUAGAGGGCAUGAGC (SEQ ID NO: 81) |
| hsa-miR-8075 | CCUUGCUGAUGGCAGAUGUCGGAUCUGCCUCGCUUAUACGUG |
| | CCCUUGCUGAUGGCAGAUGUCGGGUCUGCCUCGCUUAU |
| | (SEQ ID NO: 82) |
| hsa-miR-8089 | AAGGAGCACUCACUCCAAUUUCCCUGGACUGGGGGCAGGCUG |
| | CCACCUCCUGGGGACAGGGGAUUGGGGCAGGAUGUUCCAG |
| | (SEQ ID NO: 83) |
| hsa-miR-892b | UGCAAUGCCCUACUCAGAAAGGUGCCAUUUAUGUAGAUUUUA |
| | UGUCACUGGCUCCUUUCUGGGUAGAGCAAGGCUCA |
| | (SEQ ID NO: 84) |
| hsa-miR-936 | ACAGUAGAGGGAGGAAUCGCAG (SEQ ID NO: 85) |
| hsa-miR-937-5p | GUGAGUCAGGGUGGGGCUGG (SEQ ID NO: 86) |
| scaRNA17 | AGAGGCUUGGGCCGCCGAGCUGGACCCGGACCGGUUUUGGGU |
| | ACUGUACUGGGGGCAGGGCAGAGAGGG (SEQ ID NO: 87) |
| U6 | GUGCUCGCUUCGGCAGCACAUAUACUAAAAUUGGAACGAUAC |
| | AGAGAAGAUUAGCAUGGCCCCUGCGCAAGGAUGACACGCAAA |
| | UUCGUGAAGCGUUCCAUAUUUU (SEQ ID NO: 88) |

### Example 1: Expression of Human Mature miRNAs by qPCR in Sample Cohort of 45 AD Patients and 182 Controls

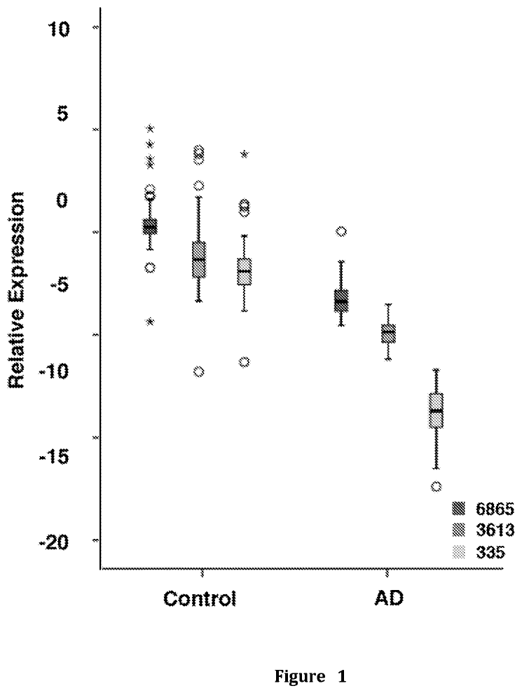

The mean log fold change for hsa-miR-335-5p, hsa-miR-3613-3p and hsa-miR-6865-3p PARKmiRs between AD patients and healthy controls are illustrated in FIG. 1. Specificity of PARKmiRs. Plot for qRT-PCR data showing distinct expression (log) patterns observed for PARKmiRs in 50 AD patient serum samples as compared to 182 control serum samples.

### Example 2: Analyses of PARKmiR Combinations, Hsa-miR-335-5p/Hsa-miR-3613-3p, Hsa-miR-3613-3p/Hsa-miR-6865-3p and Hsa-miR-335-5p/Hsa-miR-6865-3p in Sample Cohort of 45 AD Patients and 182 Controls

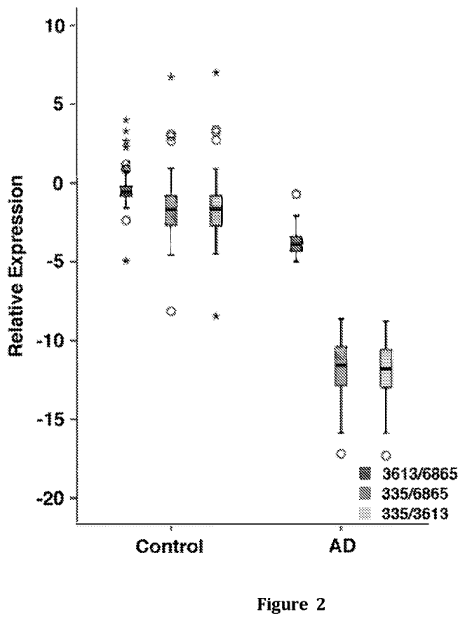

The qPCR technique of Example 1 was used to identify potential diagnostic biomarkers. It was determined that combinations of PARKmiRs show high predictability for AD diagnosis. The results of the model with hsa-miR-335-5p/hsa-miR-6865-3p, hsa-miR-335-5p/hsa-miR-3613-3p and hsa-miR-6865-3p/hsa-miR-3613-3p between AD patients and healthy controls are illustrated in FIG. 2. Specificity of PARKmiR combinations. Plot for qRT-PCR data showing distinct expression (log) patterns observed for P ARKmiR combinations in 50 AD patient serum samples as compared to 182 control serum samples.

### Example 3

It is evidenced that any combination of three or more microRNAs from the list of 85 identified miRNAs can be used to diagnose the occurrence of AD in patients.

### Example 4

Measurement of levels of a combination of two or more miRNAs in serum from patients can assist in distinctly differentiating between a potential AD patient and a healthy individual. A serum sample is obtained from blood withdrawn from patients suspected of AD. The serum is used for total microRNA isolation and enrichment. This RNA would then be tested using qPCR to measure the levels of any two or more of the 85 miRNAs mentioned in Example 1, or any one of three miRNAs mentioned in Examples 5-7. Detectable levels of any two or more of the 85 miRNAs or any one of the three miRNAs confirms the patient has AD. If desired, other sample fluids may be utilized, including plasma, venous or arterial blood, or CSF samples withdrawn by lumbar puncture. Such plasma, blood or CSF samples are processed as discussed above regarding serum, e.g., so as to provide a sample for processing and evaluation outside the human or animal body. It will be understood that measurement of more than two miRNAs in combination or a set of combinations used in a test matrix may desirably increase the accuracy of AD diagnosis. Following diagnosis, the result is then communicated to the patient.

### Example 5

Since a combination of miRNA can be used for diagnosis it may be advisable to test all the candidates to eliminate any cohort-based variation. It is understood that any detectable amounts of relevant miRNA will indicate AD pathology. However, those of ordinary skill in the art recognize it may be clinically helpful to use values of 45 v 182 samples to set an artificial threshold for diagnosis. Differential miRNA levels can be used to develop diagnostic biomarker kits that can be used by clinicians in diagnosis as well as in clinical trials. In this study the presence and quantification of miRNA from serum was determined by qRT-PCR which amplifies and quantifies the RNA is question. Other suitable techniques known to those of ordinary skill herein may be alternatively utilized, including use of labeled antisense sequences and labeled antibodies. Suitable antibodies are preferentially selective, referring to a binding reaction between two molecules that is typically more than 10 to 100 times background molecular associations under measurement conditions. Thus, under designated immunoassay conditions, the specified antibodies bind to a particular miRNA sequence, thereby identifying its presence. Specific binding to an antibody under such conditions requires an antibody that is selected for its specificity for a particular miRNA. For example, antibodies raised against a particular miRNA can be selected by subtracting out antibodies that cross-react with other molecules. A variety of immunoassay formats may be used to select antibodies specifically immunoreactive with a particular miRNA including solid-phase ELISA immunoassay's (see, e.g., Harlow & Lane, Antibodies, A Laboratory Manual (1988) for a description of immunoassay formats and conditions that can be used to determine specific immunoreactivity). Methods for determining whether two molecules specifically interact are disclosed therein, and methods of determining binding affinity and specificity are well known in the art (see, for example, Harlow and Lane, Antibodies: A laboratory manual (Cold Spring Harbor Laboratory Press, 1988): Friefelder, “Physical Biochemistry: Applications to biochemistry and molecular biology” (W.H. Freeman and Co. 1976)). The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)2, Fab, Fv and rIgG). See also, Pierce Catalog and Handbook, 1994-1995 (Pierce Chemical Co., Rockford, IL). See also, e.g., Kuby, J., Immunology, 3rd Ed., W.H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science, Vol. 246 (1989) 1275-81. These and other methods of making, for example, chimeric, humanized, CDR-grafted, single chain, and bifunctional antibodies are well known to those skilled in the art (Winter and Harris, Immunol. Today, Vol. 14 (1993) 243-46; Ward et al., Nature, Vol. 341 (1989) 544-46; Harlow and Lane, supra, 1988; Hilyard et al., Protein Engineering: A practical approach (IRL Press 1992); Borrabeck, Antibody Engineering, 2d ed. (Oxford University Press 1995). Methods for producing both monoclonal and polyclonal antibodies from identified RNA sequences are well known in the art.

### Example 6

Many neurodegenerative diseases are closely related to each other when it comes to symptoms as well as pathological markers. The circulating diagnostic markers for one neurodegenerative disease can be useful for diagnosis of other disease. A method to diagnose other neurodegenerative diseases like Parkinson's Disease, Dementia with Lewy body (DLB), Amyotrophic lateral sclerosis (ALS), Multiple system atrophy (MSA), CorticoBasal Degeneration (CBD), Progressive Supranuclear Palsy (PSP) can also be developed using similar miRNA measurements of candidates mentioned above. Disease specific kits can be developed similar to that mentioned above with various combinations of miRNAs listed in [0019].

### Example 7

The miRNAs detected in one or more combinations can regulate several proteins in the cells. Novel protein targets for AD can be discovered using these microRNAs and their combinations. The involvement of these proteins in AD etiology can be further established to target them for therapy.

### Example 8

We have experimentally confirmed the predicted regulation of LRRK2 by hsa-miR-335-5p and SNCA by hsa-miR-3613-3p in dopaminergic neuronal cell lines. Therapeutic intervention to regulate the novel targets mentioned can be achieved by RNA interference technologies.

### Example 9

Small nucleic acid molecules derived from miRNAs mentioned above will be designed to therapeutically intervene by specifically targeting genes in AD brains to achieve complete or partial remedy. The effects discussed above will be achieved for precise targeting in brain cells.

1-51. (canceled)

52. A method, comprising the steps of:
(a) providing a plasma sample from a human patient;
(b) detecting levels of SEQ ID NO: 22 and SEQ ID NO: 77 in the plasma sample;
(c) diagnosing the human patient with Alzheimer's disease when the levels of SEQ ID NO: 22 and SEQ ID NO: 77 in the plasma sample are decreased in comparison to the levels of SEQ ID NO: 22 and SEQ ID NO: 77 in control plasma samples obtained from healthy patients; and
(d) administering a cholinesterase inhibitor or an N-methyl-D-aspartate receptor antagonist to said human patient diagnosed with Alzheimer's disease.

53. The method of claim 52, further comprising detecting the level of one or more miRNA selected from the group consisting of SEQ ID NOS: 2-21, 23, 24, 25-76 and 78-86 within said plasma sample.

54. The method of claim 52, further comprising detecting the level of one or more miRNA selected from the group consisting of SEQ ID NOS: 2, 3, 5, 6, 7, 8, 9, 10, 11, 12, 13, 14, 15, 16, 24, 25, 26, 27, 28, 31, 30, 32, 33, 37, 38, 40, 42, 43, 44, 46, 47, 48, 49, 50, 51, 55, 56, 57, 58, 59, 60, 62, 63, 64, 67, 68, 71, 72, 74, 75, 78, 80, 81, 85 and 86 within said plasma sample.

55. The method of claim 52, further comprising detecting the level of one or more miRNA selected from the group consisting of SEQ ID NOS: SEQ ID NOS: 4, 17, 18, 19, 20, 21, 23, 25, 26, 29, 34, 35, 36, 39, 45, 52, 53, 54, 61, 65, 66, 69, 70, 73, 76, 79, 82, 83 and 84 within said plasma sample.

56. The method of claim 52, further comprising detecting the level of one or more miRNA selected from the group consisting of SEQ ID NOS: 15, 21, 24, 52, 54 and 55 within said plasma sample.

57. The method of claim 52, further comprising detecting the level of one or more miRNA selected from the group consisting of SEQ ID NOS: 2-21, 23, 24, 25-76 and 78-86 within said plasma sample.

58. The method of claim 52, further comprising detecting the level of one or more miRNA selected from the group consisting of SEQ ID NOS: 2, 3, 5, 6, 7, 8, 9, 10, 11, 12, 13, 14, 15, 16, 24, 25, 26, 27, 28, 31, 30, 32, 33, 37, 38, 40, 42, 43, 44, 46, 47, 48, 49, 50, 51, 55, 56, 57, 58, 59, 60, 62, 63, 64, 67, 68, 71, 72, 74, 75, 78, 80, 81, 85 and 86 within said plasma sample.

59. The method of claim 52, further comprising detecting the level of one or more miRNA selected from the group consisting of SEQ ID NOS: 4, 17, 18, 19, 20, 21, 23, 25, 26, 29, 34, 35, 36, 39, 45, 52, 53, 54, 61, 65, 66, 69, 70, 73, 76, 79, 82, 83 and 84 within said plasma sample.

60. The method of claim 52, further comprising detecting the level of one or more miRNA selected from the group consisting of SEQ ID NOS: 15, 21, 24, 52, 54 and 55 within said plasma sample.

61. The method of claim 53, wherein said one or more miRNA comprises SEQ ID NO: 15.

62. The method of claim 53, wherein said one or more miRNA comprises SEQ ID NO: 21.

63. The method of claim 53, wherein said one or more miRNA comprises SEQ ID NO: 24.

64. The method of claim 53, wherein said one or more miRNA comprises SEQ ID NO: 52.

65. The method of claim 53, wherein said one or more miRNA comprises SEQ ID NO: 54.

66. The method of claim 53, wherein said one or more miRNA comprises SEQ ID NO: 55.

67. The method of claim 52, wherein said cholinesterase inhibitor is donepezil, galantamine or rivastigmine.

68. The method of claim 52, wherein said N-methyl-D-aspartate receptor antagonist is memantine.

69. The method of claim 52, wherein said one or more miRNA comprises SEQ ID NO: 25.

70. The method of claim 69, further comprising detecting the level of one or more miRNA selected from the group consisting of SEQ ID NOS: 2-21, 23, 24, 26-76 and 78-86 within said plasma sample.

71. The method of claim 69, further comprising detecting the level of at least one or more miRNA selected from the group consisting of SEQ ID NOS: 2, 3, 5, 6, 7, 8, 9, 10, 11, 12, 13, 14, 15, 16, 24, 26, 27, 28, 31, 30, 32, 33, 37, 38, 40, 42, 43, 44, 46, 47, 48, 49, 50, 51, 55, 56, 57, 58, 59, 60, 62, 63, 64, 67, 68, 71, 72, 74, 75, 78, 80, 81, 85 and 86 within said plasma sample.

72. The method of claim 69, further comprising detecting the level of at least one or more miRNA selected from the group consisting of SEQ ID NOS: SEQ ID NOS: 4, 17, 18, 19, 20, 21, 23, 26, 29, 34, 35, 36, 39, 45, 52, 53, 54, 61, 65, 66, 69, 70, 73, 76, 79, 82, 83 and 84 within said plasma sample.

73. The method of claim 69, further comprising detecting the level of at least one or more miRNA selected from the group consisting of SEQ ID NOS: 15, 21, 24, 52, 54 and 55 within said plasma sample.

74. The method of claim 69, further comprising detecting the level of at least two or more miRNAs selected from the group consisting of SEQ ID NOS: 2-21, 23, 24, 26-76 and 78-86 within said plasma sample.

75. The method of claim 69, further comprising detecting the level of at least two or more miRNAs selected from the group consisting of SEQ ID NOS: 2, 3, 5, 6, 7, 8, 9, 10, 11, 12, 13, 14, 15, 16, 24, 26, 27, 28, 31, 30, 32, 33, 37, 38, 40, 42, 43, 44, 46, 47, 48, 49, 50, 51, 55, 56, 57, 58, 59, 60, 62, 63, 64, 67, 68, 71, 72, 74, 75, 78, 80, 81, 85 and 86 within said plasma sample.

76. The method of claim 69, further comprising detecting the level of at least two further miRNAs selected from the group consisting of SEQ ID NOS: 4, 17, 18, 19, 20, 21, 23, 26, 29, 34, 35, 36, 39, 45, 52, 53, 54, 61, 65, 66, 69, 70, 73, 76, 79, 82, 83 and 84 within said plasma sample.

77. The method of claim 69, further comprising detecting the levels of at least two or more miRNAs selected from the group consisting of SEQ ID NOS: 15, 21, 24, 52, 54 and 55 within said plasma sample.

78. The method of claim 70, wherein said at least one or more miRNA comprises SEQ ID NO: 15.

79. The method of claim 70, wherein said at least one or more miRNA comprises SEQ ID NO: 21.

80. The method of claim 70, wherein said at least one or more miRNA comprises SEQ ID NO: 24.

81. The method of claim 70, wherein said at least one or more miRNA comprises SEQ ID NO: 52.

82. The method of claim 70, wherein said at least one or more miRNA comprises SEQ ID NO: 54.

83. The method of claim 70, wherein said at least one or more miRNA comprises SEQ ID NO: 55.

84. The method of claim 69, wherein said cholinesterase inhibitor is donepezil, galantamine or rivastigmine.

85. The method of claim 69, wherein said N-methyl-D-aspartate receptor antagonist is memantine.
